(19) United States
(12) Patent Application Publication
(10) Pub. No.: US 2022/0415450 A1
(43) Pub. Date: Dec. 29, 2022

(54) SYSTEM AND METHOD FOR ESTIMATING SOLUBILITY

(71) Applicant: SAMSUNG ELECTRONICS CO., LTD., SUWON-SI (KR)

(72) Inventors: THANH CUONG NGUYEN, HWASEONG-SI (KR); DAEKEON KIM, HWASEONG-SI (KR); JIYOUNG PARK, SUWON-SI (KR); HYUNWOO KIM, SEONGNAM-SI (KR); SEUNGMIN LEE, HWASEONG-SI, GYEONGGI-DO (KR); INKOOK JANG, SEOUL (KR); SUKKOO HONG, SUWON-SI (KR)

(21) Appl. No.: 17/850,722

(22) Filed: Jun. 27, 2022

(30) Foreign Application Priority Data
Jun. 29, 2021 (KR) 10-2021-0084874
Aug. 23, 2021 (KR) 10-2021-0111204

(51) Int. Cl. G16C 20/30; G16C 20/70

(52) U.S. Cl. CPC G16C 20/30; G16C 20/70

(57) ABSTRACT

A method of estimating solubility includes obtaining input data representing a chemical structure of a target material; generating at least one descriptor based on the input data; obtaining at least one solubility parameter by providing the at least one descriptor to a machine learning model trained based on chemical structures and sample solubility parameters of sample materials; and calculating the solubility based on the at least one solubility parameter, wherein the at least one descriptor includes at least one of a zero-dimensional descriptor, a one-dimensional descriptor, a two-dimensional descriptor, or a three-dimensional descriptor, each representing the chemical structure of the target material.

### CROSS-REFERENCE TO RELATED APPLICATIONS

This application is based on and claims priority under 35 U.S.C. § 119 to Korean Patent Application No. 10-2021-0084874, filed on Jun. 29, 2021 in the Korean Intellectual Property Office, and Korean Patent Application No. 10-2021-0111204, filed on Aug. 23, 2021 in the Korean Intellectual Property Office, the disclosures of which are incorporated by reference herein in their entirety.

### BACKGROUND

### 1. Field

The present disclosure relates to the solubility of a solute in a solvent, and more particularly, to a system and method for estimating solubility.

### 2. Description of the Related Art

Solubility may indicate a characteristic of a solute dissolving in a solvent. Different solutes may have different solubilities in one solvent, and a solute may have different solubilities in different solvents. The solubility of a solute and solvent may be used as significant indicators for determining the use of a solution. Solubility may be detected through an experiment, but it may be practically difficult to repeat experiments for detecting solubility corresponding to each of many combinations of various solutes and solvents when the purpose of the experiments is to derive (e.g., identify) a solute and a solvent having desired solubility.

### SUMMARY

The teachings herein describe a system and method for quickly and accurately estimating solubility.

According to an aspect of the present disclosure, a method of estimating solubility includes obtaining input data representing a chemical structure of a target material; generating at least one descriptor based on the input data; obtaining at least one solubility parameter by providing the at least one descriptor to a machine learning model trained based on chemical structures and sample solubility parameters of sample materials; and calculating the solubility based on the at least one solubility parameter. The at least one descriptor includes at least one of a zero-dimensional descriptor, a one-dimensional descriptor, a two-dimensional descriptor, or a three-dimensional descriptor, each representing the chemical structure of the target material.

According to another aspect of the present disclosure, a system includes at least one processor; and a non-transitory storage medium storing instructions allowing the at least one processor to perform operations for solubility estimation when the instructions are executed by the at least one processor. The operations include an operation of obtaining input data representing a chemical structure of a target material; an operation of generating at least one descriptor based on the input data; an operation of obtaining at least one solubility parameter by providing the at least one descriptor to a machine learning model trained based on chemical structures and sample solubility parameters of sample materials; and an operation of calculating the solubility based on the at least one solubility parameter. The at least one descriptor includes at least one of a zero-dimensional descriptor, a one-dimensional descriptor, a two-dimensional descriptor, or a three-dimensional descriptor, each representing the chemical structure of the target material.

According to a further aspect of the present disclosure, a method of estimating solubility includes generating a machine learning model trained to derive at least one solubility parameter from at least one descriptor defining a chemical structure of a material. The generating of the trained machine learning model includes obtaining training data with respect to an attribute of a sample material; generating a plurality of sample descriptors based on the training data; extracting at least one sample solubility parameter of the sample material from the training data; and training the machine learning model based on the plurality of sample descriptors and the at least one sample solubility parameter. The plurality of sample descriptors include at least one of a zero-dimensional descriptor, a one-dimensional descriptor, a two-dimensional descriptor, or a three-dimensional descriptor, each representing a chemical structure of the sample material.

### BRIEF DESCRIPTION OF THE DRAWINGS

Embodiments of the inventive concept(s) described herein will be more clearly understood from the following detailed description taken in conjunction with the accompanying drawings in which:

FIG. 1 illustrates a flowchart of a method of estimating solubility, according to an example embodiment;

FIG. 2 is a diagram illustrating a procedure for manufacturing an integrated circuit through semiconductor processes, according to an example embodiment;

FIG. 3 is a diagram illustrating a photoresist material according to an example embodiment;

FIG. 4A, FIG. 4B and FIG. 4C are diagrams of the chemical structures of materials according to an example embodiment;

FIG. 5 is a diagram illustrating examples of descriptors according to an example embodiment;

FIG. 6 is a diagram illustrating a machine learning model according to an example embodiment;

FIG. 7 illustrates a flowchart of a method of estimating solubility, according to an example embodiment;

FIG. 8 illustrates a flowchart of a method of estimating solubility, according to an example embodiment;

FIG. 9 illustrates a flowchart of a method of estimating solubility, according to an example embodiment;

FIG. 10 illustrates a flowchart of a method of estimating solubility, according to an example embodiment;

FIG. 11 is a diagram illustrating the structure of a database representing a material, according to an example embodiment;

FIG. 12 illustrates a flowchart of a method of estimating solubility, according to an example embodiment;

FIG. 13A, FIG. 13B and FIG. 13C illustrate graphs of solubility parameters according to an example embodiment; and

FIG. 14 is a block diagram illustrating a computing system according to an example embodiment.

### DETAILED DESCRIPTION OF THE EMBODIMENTS

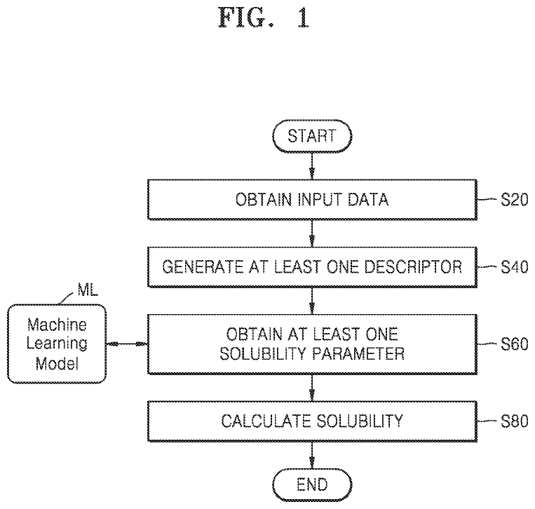

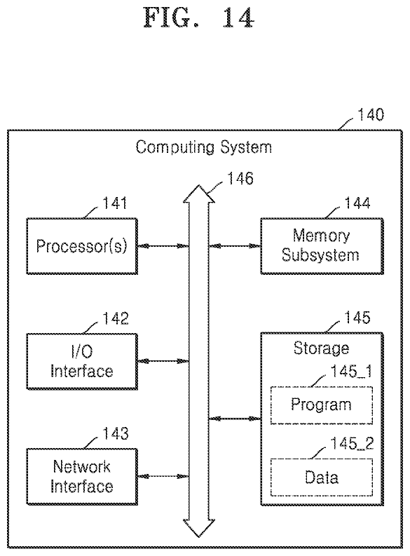

FIG. 1 illustrates a flowchart of a method of estimating solubility, according to an example embodiment. The method of FIG. 1 may be performed by a computing system. An example of a computing system is the computing system 140 shown in FIG. 14 and described below. Referring to FIG. 1, the method may include a plurality of operations S20, S40, S60, and S80.

The operations of methods described below may be performed by appropriate units, e.g., various hardware and/or software components, circuits, and/or modules. Software may include an ordered list of executable instructions for implementing logical functions and may be used by an instruction execution system, apparatus or device or embodied in any relevant processor-readable medium. An example of an instruction execution system, apparatus or device is a system, apparatus or device which includes one or more single-core processor and/or multi-core processor which execute(s) executable instructions.

The steps or blocks and functions of a method or algorithm described below may be embodied directly in hardware, a software module executed by a processor, or a combination thereof. When functions are implemented by software, the functions may be stored as at least one instruction or code in a non-transitory tangible computer-readable medium. A software module may be in random access memory (RAM), flash memory, read-only memory (ROM), electrically programmable ROM (EPROM), electrically erasable programmable ROM (EEPROM), registers, a hard disk, a removable disk, a CD-ROM, or any other form of storage medium.

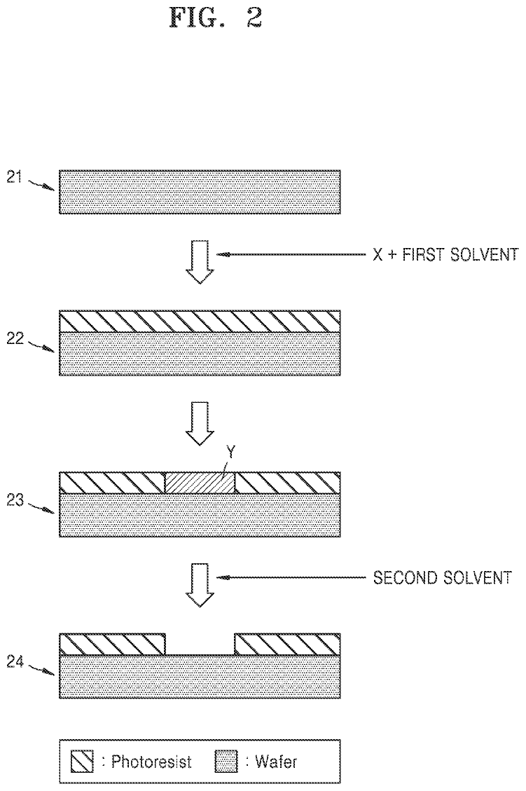

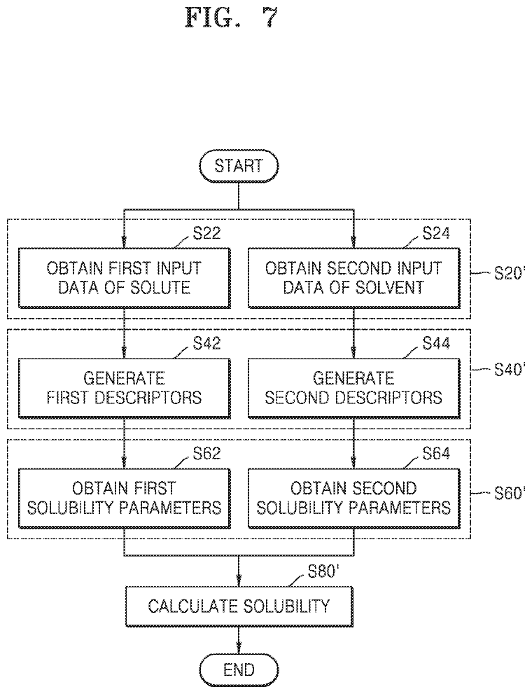

Referring to FIG. 1, input data may be obtained in operation S20. The input data may represent the chemical structure of a target material. The target material may be related to solubility to be estimated and may be a solvent or a solute. As described below with reference to FIG. 7, when a solute is a composite, the target material may be each of the materials of the composite. For example, as described below with reference to FIG. 2, the target material may include a material of photoresist or a material of a solvent dissolving the photoresist.

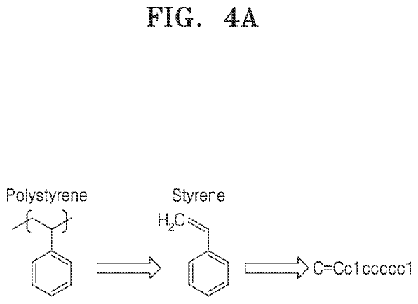

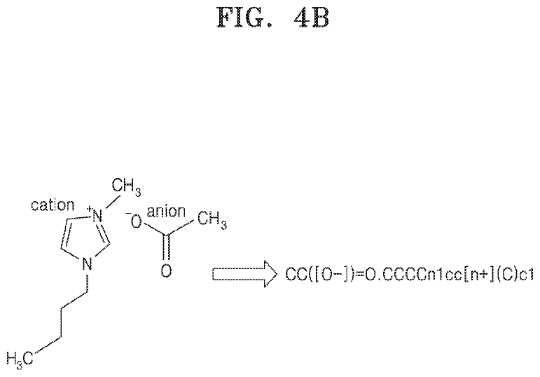

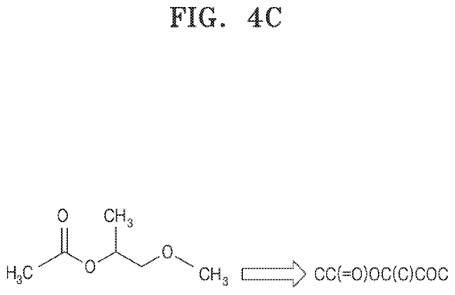

The input data may have a form representing the chemical structure of the target material. In some embodiments, the input data may include a string including a series of characters, which defines the chemical structure of the target material. For example, the input data may include a string expressed based on simplified molecular-input line-entry system (SMILES) code, smiles arbitrary target specification (SMARTS) code, international chemical identifier (InChi) code, or the like. Examples of a string are described with reference to FIG. 4A, FIG. 4B and FIG. 4C below.

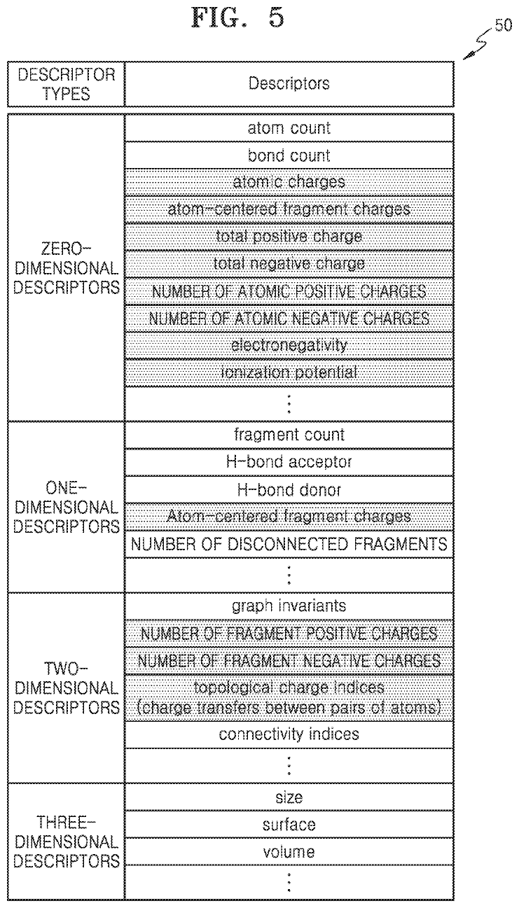

At least one descriptor may be generated in operation S40. The descriptor may have a value representing a characteristic of the target material, and there may be various descriptors corresponding to the target material. For example, as described below with reference to FIG. 5, descriptors may be classified into zero-dimensional descriptors, one-dimensional descriptors, two-dimensional descriptors, and three-dimensional descriptors. A descriptor may be constituted of at least one number and used as an input of a machine learning model ML. Two-dimensional descriptors may be referred to as topological descriptors, and three-dimensional descriptors may be referred to as geometric descriptors.

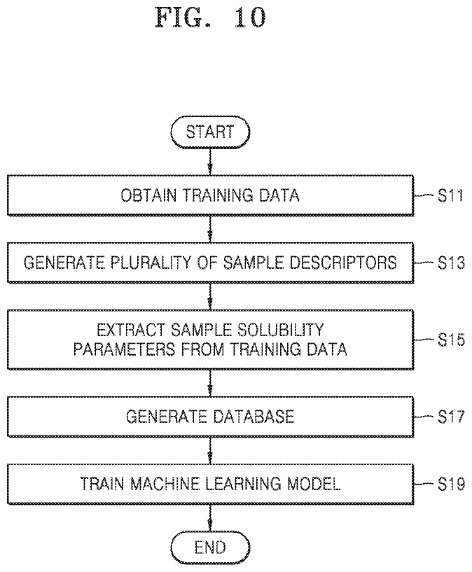

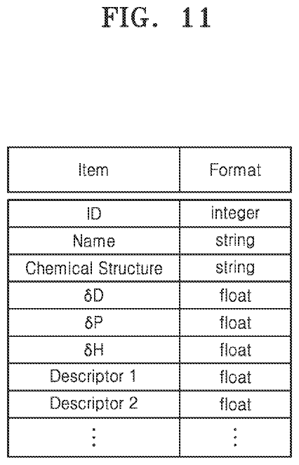

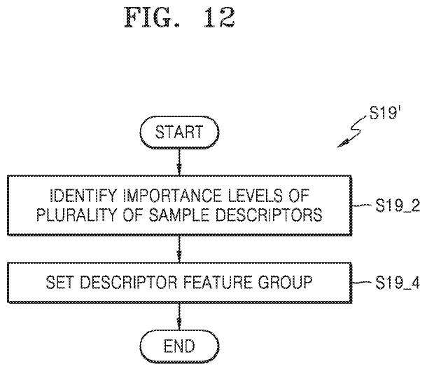

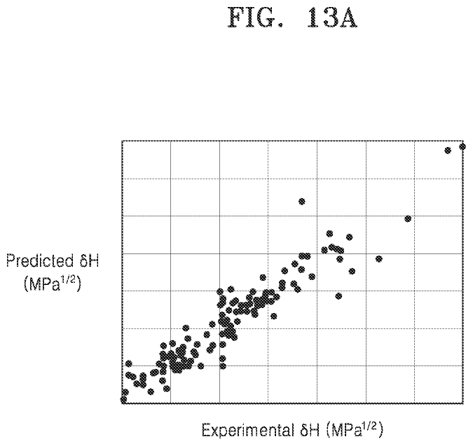

At least one solubility parameter may be obtained in operation S60. For example, as shown in FIG. 1, the at least one solubility parameter obtained at S60 may be obtained from the machine learning model ML. The machine learning model ML may have been trained on training data, and the training data may include information (i.e., descriptors) about the chemical structures of sample materials and solubility parameters. Solubility parameters described by information included in training data may be referred-to herein as sample solubility parameters. Training of the machine learning model ML is described with reference to FIG. 10, FIG. 11 and FIG. 12 below. The machine learning model ML may be trained based on chemical structures of sample materials and sample solubility parameters. In some embodiments, the machine learning model ML may be implemented by a dedicated hardware device, e.g., a neural processing unit (NPU), a tensor processing unit (TPU), or a neural engine, which is designed to implement the machine learning model ML. In some embodiments, the machine learning model ML may be implemented by a general-purpose programmable hardware device, e.g., a central processing unit (CPU), a digital signal processor (DSP), or a graphics processing unit (GPU).

The machine learning model ML may have a structure that is trained on training data. For example, the machine learning model ML may include an artificial neural network, a decision tree, a support vector machine, a Bayesian network, and/or a genetic algorithm. Hereinafter, an artificial neural network is mainly referred to in the descriptions of the machine learning model ML below, but embodiments are not limited thereto. As a non-limiting example, the artificial neural network may include a convolution neural network (CNN), a region with CNN (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzmann machine (RBM), a fully convolutional network, a long short-term memory (LSTM) network, or a classification network.

Solubility may be calculated in operation S80. For example, the solubility may be calculated based on the at least one solubility parameter obtained in operation S60. To determine the capacity of a solute to dissolve in a solvent, Gibbs free energy of mixing may be used. The Gibbs free energy of mixing may be calculated based on the variation of enthalpy of mixing, absolute temperature, and the variation of entropy of mixing. The Gibbs free energy of mixing that is less than zero may indicate that a solute dissolves well in a solvent. The Gibbs free energy of mixing that is greater than zero may indicate that a solute does not dissolve well in a solvent. The Gibbs free energy of mixing may be related to a dispersion force, a dipolar intermolecular force, and a hydrogen bond; and a distance R between solubility parameters in a Hansen space may be defined as Equation 1.

R²=4(δD_{A}−δD_{B})²+(δP_{A}−δP_{B})²+(SH_{A}−δH_{B})² [Equation 1]

In Equation 1, δD_{A} is a dispersion force (or energy by a dispersion force) between solute molecules. δP_{A} is a dipolar intermolecular force (or energy by a dipolar intermolecular force) between the solute molecules. δH_{A} is a force (or energy) of a hydrogen bond between the solute molecules. δD_{B} is a dispersion force (or energy by a dispersion force) between solvent molecules. δP_{B} is a dipolar intermolecular force (or energy by a dipolar intermolecular force) between the solvent molecules. δH_{B} is a force (or energy) of a hydrogen bond between the solvent molecules. δD_{A}, δP_{A}, δH_{A}, δD_{B}, δP_{B}, and δH_{B} may be collectively referred to as Hansen solubility parameters. At least one solubility parameter may include a dispersion force parameter, a polar force parameter, and/or a hydrogen bond force parameter as Hansen solubility parameters. δD_{A}, δP_{A}, and δH_{A} may be referred-to herein as first solubility parameters. δD_{B}, δP_{B}, and δH_{B} may be referred-to herein as second solubility parameters. Solubility may be proportional to the reciprocal (1/R) of the distance R between solubility parameters. For example, solubility may be calculated as the reciprocal of R, i.e., 1/R. To calculate solubility based on Equation 1, solubility parameters of a solute and solubility parameters of a solvent may be obtained, and solubility may be calculated based on the obtained solubility parameters.

As described above, solubility may be quickly and accurately estimated using the machine learning model ML that is trained to output a solubility parameter from the chemical structure of a material, and accordingly, a solute and/or a solvent that is required by an application may be easily determined. In addition, because of easy determination of the solute and/or the solvent, the efficiency of applications using a solution may be enhanced. For example, as described with reference to FIG. 2 below, when the solubility of a solution used in semiconductor processes is accurately estimated, the semiconductor processes may be efficiently constructed. Moreover, integrated circuits may be exactly manufactured according to the design through the semiconductor processes, and the yield of integrated circuits may be increased.

FIG. 2 is a diagram illustrating a procedure for manufacturing an integrated circuit through semiconductor processes, according to an example embodiment. In detail, FIG. 2 shows cross-sectional views of structures formed by semiconductor processes. The integrated circuit may include an analog signal processor, a DSP, or a combination thereof.

The semiconductor processes may include various sub-processes of forming patterns of the integrated circuit. For example, semiconductor processes may include photolithography, which may refer to a process of forming a pattern by transferring a geometric pattern from a photomask to a photosensitive chemical photoresist using light. Photoresist may include a positive photoresist, of which an exposed portion is soluble in developer, and a negative photoresist, of which an unexposed portion is soluble in developer. FIG. 2 shows an example of photolithography of forming a pattern using a positive photoresist. As explained below with respect to FIG. 2, a solvent may be used as a developer to dissolve a material.

Referring to FIG. 2, a wafer may be prepared in a first state 21. For example, the wafer may include patterns formed by at least one sub-process.

A positive photoresist may be applied to the wafer in a second state 22. As shown in FIG. 2, to apply the positive photoresist to the wafer, a photoresist material X as a solute may be dissolved in a first solvent, and the photoresist material X and a solvent constituted of the first solvent may be applied as the positive photoresist to the wafer. Accordingly, the photoresist material X and the first solvent may be required to provide high solubility so that the photoresist material X is uniformly applied to the wafer. In some embodiments, the positive photoresist may be applied to an oxide layer by spin coating. In some embodiments, after the application of the positive photoresist, the wafer may be heated to remove an excess solvent.

A photomask may be aligned above in the second state 22, and light, e.g., extreme ultraviolet (EUV) light, may be radiated to the aligned photomask. Accordingly, the positive photoresist exposed to the light, as shown in FIG. 2, may be chemically modified in a third state 23, and a material Y may be formed.

Developer may be provided in the third state 23, and accordingly, a portion of a photoresist layer, which has been irradiated with the light, i.e., the material Y, may be dissolved in the developer and removed in a fourth state 24. A process of removing the portion of the photoresist layer, which has been chemically modified by light, may be referred to as developing. As shown in FIG. 2, a second solvent may be used as the developer, and the material Y and the second solvent may be required to provide high solubility to remove the material Y. After the fourth state 24, etching and cleaning may be subsequently performed.

As described above, the photoresist material X may be required to dissolve well in the first solvent, and the material Y modified from the photoresist material X by light may be required to dissolve well in the second solvent. Accordingly, to exactly form a designed pattern, it may be important to determine the photoresist material X, the first solvent, and the second solvent. To determine the photoresist material X, the first solvent, and the second solvent, the method of estimating solubility described above with reference to FIG. 1 may be used, and accordingly, the photoresist material X, the first solvent, and the second solvent may be accurately and easily determined.

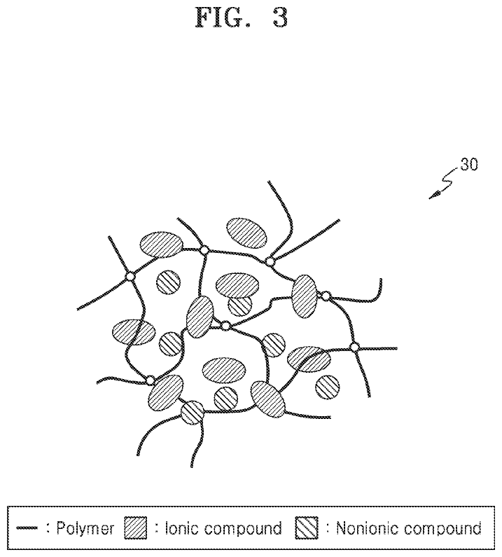

FIG. 3 is a diagram illustrating a photoresist material 30 according to an example embodiment. As described above with reference to FIG. 2, the photoresist material 30 may be used to form a pattern of an integrated circuit in semiconductor processes.

Referring to FIG. 3, the photoresist material 30 may include a polymer, an ionic compound, and a nonionic compound. For example, the polymer may take up the greatest proportion in the photoresist material 30 and have a network structure of at least several nanometers. The ionic compound may take up the second greatest proportion in the photoresist material 30 and may be constituted of cation-anion pairs. In some embodiments, the ionic compound may include an ionic organic compound. The nonionic compound may take up the least proportion in the photoresist material 30 and may be included in a solvent, e.g., the first solvent in FIG. 2, which dissolves the photoresist material 30. In some embodiments, the nonionic compound may include a covalent ionic compound. As described below with reference to the drawings, the solubility of a solvent and a solute, which is constituted of various materials, e.g., the photoresist material 30, including an ionic compound may be estimated.

FIG. 4A, FIG. 4B and FIG. 4C are diagrams of the chemical structures of materials according to an example embodiment. In detail, FIG. 4A shows an example of the polymer in FIG. 3, FIG. 4B shows an example of the ionic compound in FIG. 3, and FIG. 4C shows an example of the nonionic compound in FIG. 3. FIG. 4A, FIG. 4B and FIG. 4C are described with reference to FIG. 1 below.

As described above with reference to FIG. 1, the input data may include the string representing the chemical structure of the target material. FIG. 4A, FIG. 4B and FIG. 4C show strings representing chemical structures based on SMILES. For example, as shown in FIG. 4A, polystyrene, as an example of the polymer, may include styrene, which may be expressed as string “C—Cc1ccccc1”. As shown in FIG. 4B, 1-butyl-3-methilimidazolium acetate, as an example of the ionic compound, may be expressed as string “CC([O—])═O.CCCCn1cc[n+](C)c1” As shown in FIG. 4C, propylene glycol methyl ether acetate, as an example of the nonionic compound, may be expressed as string “CC(═O)OC(C)COC”.

FIG. 5 is a diagram illustrating examples of descriptors according to an example embodiment. In detail, a table 50 of FIG. 5 shows descriptor types and descriptors included in each descriptor type. As described above with reference to FIG. 1, at least one descriptor may be generated from the input data representing the chemical structure of the target material. For example, as described above with reference to FIG. 4A, FIG. 4B and FIG. 4C, at least one descriptor may be generated from a string representing the chemical structure of the target material. In some embodiments, only some of the descriptors in FIG. 5 may be generated from the string. In some embodiments, descriptors that are not shown in FIG. 5 may be generated from the string. As described below, each of the descriptors in FIG. 5 may be constituted of at least one number and used as an input of the machine learning model ML in FIG. 1.

Referring to FIG. 5, descriptors may include zero-dimensional descriptors, one-dimensional descriptors, two-dimensional descriptors, and three-dimensional descriptors. Zero-dimensional descriptors may include an atom count, a bond count, atomic charges, atom-centered fragment charges, total positive charge, total negative charge, the number of atomic positive charges, the number of atomic negative charges, electronegativity, ionization potential, and so on. One-dimensional descriptors may include a fragment count, a hydrogen bond acceptor (i.e., a H-bond acceptor), a hydrogen bond donor (i.e., a H-bond donor), atom-centered fragment charges, the number of disconnected fragments, and so on. Two-dimensional descriptors may include at least one of graph invariants, the number of fragment positive charges, the number of fragment negative charges, topological charge indices, connectivity indices, and so on. The topological charge indices refer to the number of charge transfers between pairs of atoms. Three-dimensional descriptors may include a size, a surface, a volume, and so on.

In some embodiments, descriptors may include electrostatic descriptors. For example, as shown in the shaded portions in the table 50 of FIG. 5, the atom count, the atom-centered fragment charges, the total positive charge, the total negative charge, the number of atomic positive charges, the number of atomic negative charges, the electronegativity, and the ionization potential among the zero-dimensional descriptors may correspond to electrostatic descriptors; the atom-centered fragment charges among the one-dimensional descriptors may correspond to electrostatic descriptors; and the number of fragment positive charges, the number of fragment negative charges, and the topological charge indices among the two-dimensional descriptors may correspond to electrostatic descriptors. Accordingly, the solubility of an ionic material such as an ionic compound or the solubility of a material including an ionic material may be estimated.

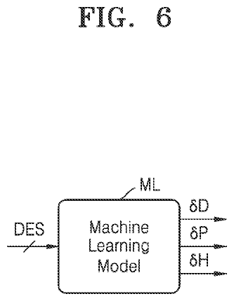

FIG. 6 is a diagram illustrating the machine learning model ML according to an example embodiment. As described above with reference to FIG. 1, the machine learning model ML may have been trained on training data so as to output a solubility parameter.

Referring to FIG. 6, at least one descriptor DES may be provided to the machine learning model ML. For example, the at least one descriptor DES among the descriptors in the table 50 may be provided to the machine learning model ML and may include a series of numbers. When at least two descriptors are provided to the machine learning model ML, the descriptors may be arranged in a predefined order, i.e., the order of descriptors provided to the machine learning model ML during the training of the machine learning model ML, and provided to the machine learning model ML in the predefined order.

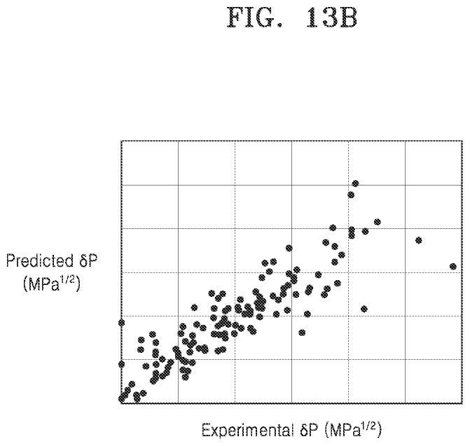

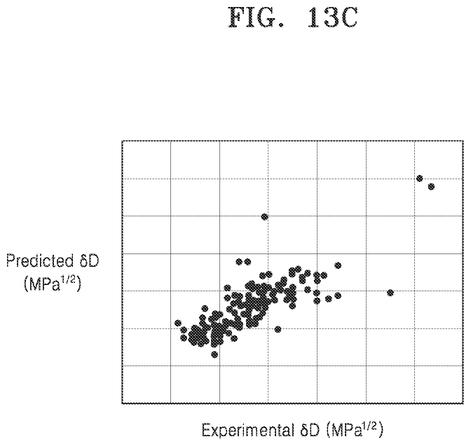

The machine learning model ML may generate Hansen solubility parameters δD, δP, and δH from the descriptor DES. For example, the machine learning model ML may have states, e.g., network topology, bias, and a weight, which are determined through training, and generate the Hansen solubility parameters δD, δP, and δH by processing the descriptor DES. When a solute is a composite of at least two materials, one or more solubility parameter may be calculated based on a weighted sum of at least two solubility parameters respectively corresponding to the at least two materials. When the solvent is a mixture of at least two solvents, one or more solubility parameters may be calculated based on a weighted sum of at least two solubility parameters corresponding to the at least two solvents. As described above with reference to FIG. 1, δD indicates a dispersion force between molecules of a target material, δP indicates a dipolar intermolecular force (or a polar force) between molecules of the target material, and δH indicates the force of an H-bond between molecules of the target material. As described below with reference to FIG. 13A, FIG. 13B and FIG. 13C, the machine learning model ML may generate Hansen solubility parameters highly similar to Hansen solubility parameters derived through experiments, and accordingly, solubility may be accurately and easily estimated.

FIG. 7 illustrates a flowchart of a method of estimating solubility, according to an example embodiment. In detail, the flowchart of FIG. 7 shows examples of operations S20, S40, and S60. As shown in FIG. 7, the method of estimating solubility may include operations S20′, S40′, S60′, and S80′. FIG. 7 is described with reference to FIG. 6 below.

Referring to FIG. 7, operation S20′ may include operations S22 and S24. First input data of a solute may be obtained in operation S22, and second input data of a solvent may be obtained in operation S24. The first input data may include information, e.g., a string, representing the chemical structure of the solute. The second input data may include information, e.g., a string, representing the chemical structure of the solvent.

Operation S40′ may include operations S42 and S44. First descriptors may be generated in operation S42, and second descriptors may be generated in operation S44. For example, the first descriptors indicating attributes of the solute may be generated from the first input data obtained in operation S22, and the second descriptors indicating attributes of the solvent may be generated from the second input data obtained in operation S24.

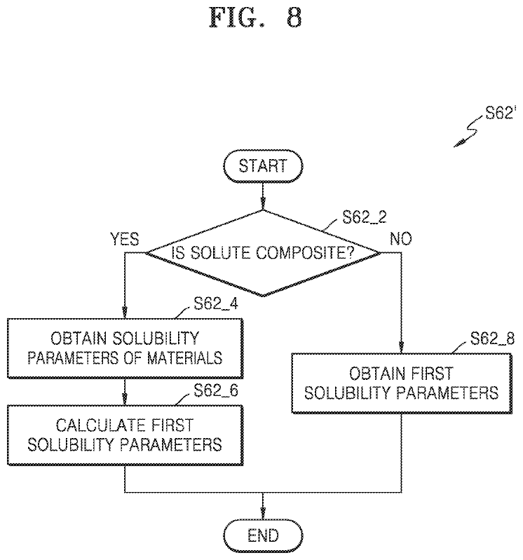

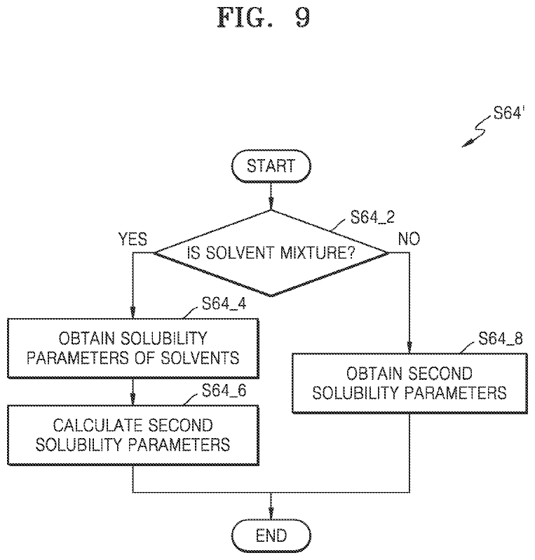

Operation S60′ may include operations S62 and S64. First solubility parameters may be obtained in operation S62, and second solubility parameters may be obtained in operation S64. For example, the first descriptors generated in operation S42 may be provided to the machine learning model ML in FIG. 6, and the machine learning model ML may output Hansen solubility parameters of the solute, i.e., the first solubility parameters, in response to the first descriptors. The second descriptors generated in operation S44 may be provided to the machine learning model ML in FIG. 6, and the machine learning model ML may output Hansen solubility parameters of the solvent, i.e., the second solubility parameters, in response to the second descriptors. An example of operation S62 is described below with reference to FIG. 8, and an example of operation S64 is described below with reference to FIG. 9.

Solubility may be calculated in operation S80′. For example, the distance R between solubility parameters in a Hansen space may be calculated from the first solubility parameters obtained in operation S62 and the second solubility parameters obtained in operation S64 using Equation 1, and the solubility of the solute in the solvent may be calculated as the reciprocal, 1/R, of the distance R.

FIG. 8 illustrates a flowchart of a method of estimating solubility, according to an example embodiment. In detail, FIG. 8 shows an example of operation S62 in FIG. 7. As described above with reference to FIG. 7, the solubility parameters of the solute, i.e., the first solubility parameters, may be obtained in operation S62′ of FIG. 8. As shown in FIG. 8, operation S62′ may include a plurality of operations S62_2, S62_4, S62_6, and S62_8.

Whether the solute is a composite may be determined in operation S62_2. As described above with reference to FIG. 3, the solute may be a composite constituted of at least two compounds. When the solute is a composite, the solubility parameters of the solute, i.e., the first solubility parameters, may be derived using a different method than the solubility parameters of a solute that is not a composite. As shown in FIG. 8, when the solute is a composite (S62_2=Yes), operations S62_4 and S62_6 may be subsequently performed. When the solute is not a composite (S62_2=No), operation S62_8 may be subsequently performed.

When the solute is a composite, solubility parameters of materials may be obtained in operation S62_4. In other words, the solute that is a composite may be constituted of at least two materials, and solubility parameters corresponding to descriptors of each of the materials may be obtained from the machine learning model ML. For example, descriptors of the polymer of the photoresist material 30 of FIG. 3 may be provided to the machine learning model ML, and the machine learning model ML may provide the solubility parameters of the polymer. In addition, descriptors of the ionic compound of the photoresist material 30 of FIG. 3 may be provided to the machine learning model ML, and the machine learning model ML may provide the solubility parameters of the ionic compound. In addition, descriptors of the nonionic compound of the photoresist material 30 of FIG. 3 may be provided to the machine learning model ML, and the machine learning model ML may provide the solubility parameters of the nonionic compound.

The first solubility parameters may be calculated in operation S62_6. In other words, the solubility parameters of the solute may be calculated based on the solubility parameters obtained in operation S62_4. For example, when the solute is constituted of N materials (e.g., compounds) (where N is an integer greater than 1), the Hansen solubility parameters δD_{A}, δP_{A}, and δH_{A} of the solute may be calculated using Equation 2.

$$δ {D}_{A} = {\sum }_{i = 1}^{N} {c}_{i} \times δ {D}_{A i} \qquad [ Equation 2 ]$$

$$δ {P}_{A} = {\sum }_{i = 1}^{N} {c}_{i} \times δ {P}_{A i}$$

$$δ {H}_{A} = {\sum }_{i = 1}^{N} {c}_{i} \times δ {H}_{A i}$$

In Equation 2, cᵢ is a proportion of the mass or volume of an i-th material in the solute, δD_{Ai} is a dispersion force of the i-th material, δP_{Ai} is a polar force of the i-th material, and δH_{Ai} is an H-bond force of the i-th material, where 1≤i≤N.

When the solute is not a composite, the first solubility parameters may be obtained in operation S62_8. For example, descriptors of the solute may be provided to the machine learning model ML, and the machine learning model ML may provide the Hansen solubility parameters δD_{A}, δP_{A}, and δH_{A} of the solute.

FIG. 9 illustrates a flowchart of a method of estimating solubility, according to an example embodiment. In detail, FIG. 9 shows an example of operation S64 in FIG. 7. As described above with reference to FIG. 7, the solubility parameters of the solvent, i.e., the second solubility parameters, may be obtained in operation S64′ of FIG. 9. As shown in FIG. 9, operation S64′ may include a plurality of operations S64_2, S64_4, S64_6, and S64_8.

Whether the solvent is a mixture may be determined in operation S64_2. The solvent may be a mixture of at least two solvents, and the solubility parameters of the mixture, i.e., the second solubility parameters, may be derived using a different method than the solubility parameters of a solvent that is not a mixture. As shown in FIG. 9, when the solvent is a mixture (S64_2=Yes), operations S64_4 and S64_6 may be subsequently performed. When the solvent is not a mixture (S64_2=No), operation S64_8 may be subsequently performed.

When the solvent is a mixture, solubility parameters of solvents may be obtained in operation S64_4. In other words, solubility parameters corresponding to descriptors of each of the solvents in the mixture may be obtained from the machine learning model ML. For example, descriptors of a first solvent in the mixture may be provided to the machine learning model ML, and the machine learning model ML may provide the solubility parameters of the first solvent. Descriptors of a second solvent in the mixture may be provided to the machine learning model ML, and the machine learning model ML may provide the solubility parameters of the second solvent.

The second solubility parameters may be calculated in operation S64_6. In other words, the second solubility parameters may be calculated based on the solubility parameters obtained in operation S64_4. For example, when the mixture is constituted of M solvents (where M is an integer greater than 1), the Hansen solubility parameters δD_{B}, δP_{B}, and δH_{B} of the mixture may be calculated using Equation 3.

$$δ {D}_{B} = {\sum }_{j = 1}^{M} {c}_{j} \times δ {D}_{Bj} \qquad [ Equation 3 ]$$

$$δ {P}_{B} = {\sum }_{j = 1}^{M} {c}_{j} \times δ {P}_{Bj}$$

$$δ {H}_{B} = {\sum }_{j = 1}^{M} {c}_{j} \times δ {H}_{Bj}$$

In Equation 3, cⱼ is a proportion of the mass or volume of a j-th solvent in the mixture, δD_{Bj} is a dispersion force of the j-th solvent, δP_{Bj} is a polar force of the j-th solvent, and δH_{Bj} is an H-bond force of the j-th solvent, where 1≤j≤M.

When the solvent is not a mixture, the second solubility parameters may be obtained in operation S64_8. For example, descriptors of the solvent may be provided to the machine learning model ML, and the machine learning model ML may provide the Hansen solubility parameters δD_{B}, δP_{B}, and δH_{B} of the solvent.

FIG. 10 illustrates a flowchart of a method of estimating solubility, according to an example embodiment. FIG. 11 is a diagram illustrating the structure of a database representing a material, according to an example embodiment. In detail, FIG. 10 illustrates a flowchart of a method of training the machine learning model ML used to estimate solubility, and FIG. 11 shows the structure of a database used to train the machine learning model ML.

Referring to FIG. 10, the method of estimating solubility may include a plurality of operations S11, S13, S15, S17, and S19. Here, the method of FIG. 10 may be referred to as an operation of generating the machine learning model ML that has been trained. The method of FIG. 10 may be performed before operation S20 in FIG. 1 in some embodiments. The method of FIG. 10 may be performed by a computing system 140 as in FIG. 14, though the training of a machine learning model ML may also or alternatively be performed by a separate but similar computing system distinct from the computing system 140 which applies the machine learning model ML according to the teachings herein.

Training data may be obtained in operation S11. For example, the training data may include information about attributes of a plurality of sample materials. For example, the training data may include information about the chemical structure of a sample material and solubility parameters of the sample material, which are obtained through experiments. In the training data, the information about the chemical structure of a sample material may have various formats. For example, the information may have a string format including a series of characters, as described above with reference to FIG. 4A, FIG. 4B and FIG. 4C.

A plurality of sample descriptors may be generated in operation S13. For example, the training data obtained in operation S 11 may include information about the chemical structures of a plurality of sample materials, and descriptors of the sample materials, i.e., sample descriptors, may be generated based on the information included in the training data. Each of sample descriptors corresponding to a single sample material may include at least one number indicating an attribute of the sample material, as described above with reference to FIG. 5.

Sample solubility parameters may be extracted from the training data in operation S15. As described above, the training data may include solubility parameters of each of the sample materials, wherein the solubility parameters are obtained through experiments, and the solubility parameters of sample materials, i.e., the sample solubility parameters, may be extracted from the training data in operation S15.

A database may be generated in operation S17. The database may be directly used for the training of the machine learning model ML. In some embodiments, the database may have the structure of FIG. 11. Referring to FIG. 11, the database about a single material may include an identifier (ID), a name, a chemical structure, Hansen solubility parameters δD, δP, and δH, and at least one descriptor. As shown in FIG. 11, the chemical structure may have a string format, and the descriptor may have a numeric format. In some embodiments, the Hansen solubility parameters δD, δP, and δH may have units of MPa^{1/2}.

The machine learning model ML may be trained in operation S19. For example, the machine learning model ML may be trained based on a method using the database generated in operation S17. The machine learning model ML may be trained based on chemical structures of sample materials and sample solubility parameters. In some embodiments, the machine learning model ML may be trained based on supervised learning using a random forest and/or a Gaussian process. The machine learning model ML may be trained based on regression learning using at least one of a random forest and a Gaussian process. An example of operation S19 is described below with reference to FIG. 12.

FIG. 12 illustrates a flowchart of a method of estimating solubility, according to an example embodiment. In detail, the flowchart of FIG. 12 shows an example of operation S19 in FIG. 10. As described above with reference to FIG. 10, the machine learning model ML may be trained in operation S19′ of FIG. 12. As shown in FIG. 12, operation S19′ may include operations S19_2 and S19_4. FIG. 12 is described with reference to FIG. 10 below.

Referring to FIG. 12, the importance levels of the sample descriptors may be identified in operation S19_2. For example, a maximum number of descriptors indicating the attributes of a sample material may be generated in operation S13 in FIG. 10. Accordingly, the database may include a plurality of descriptors, and the descriptors may be used to train the machine learning model ML. When the training of the machine learning model ML is completed, the importance levels of the inputs (i.e., the descriptors) of the machine learning model ML may be identified according to the degrees of influence on the outputs (i.e., the solubility parameters) of the machine learning model ML. For example, when coefficients multiplied by a descriptor in the machine learning model ML are low, the importance level of the descriptor may be identified as being low.

A descriptor feature group may be set in operation S19_4. For example, sample descriptors corresponding to a reference importance level or higher may be selected from among the sample descriptors based on the importance levels identified in operation S19_2, and a descriptor feature group constituted of the selected sample descriptors may be set. In some embodiments, the at least one descriptor of the target material, which is generated in operation S40 in FIG. 1, may be included in the descriptor feature group, and accordingly, the descriptors included in the descriptor feature group may be used for solubility estimation. In some embodiments, the machine learning model ML may be retrained based on descriptors included in the descriptor feature group among the descriptors corresponding to the sample materials.

FIG. 13A, FIG. 13B and FIG. 13C illustrate graphs of solubility parameters according to an example embodiment. In detail, the graph of FIG. 13A shows Hansen solubility parameters (the X axis) obtained through experiments and Hansen solubility parameters (the Y axis) derived from the machine learning model ML in terms of dispersion force. The graph of FIG. 13B shows Hansen solubility parameters (the X axis) obtained through experiments and Hansen solubility parameters (the Y axis) derived from the machine learning model ML in terms of polar force. The graph of FIG. 13C shows Hansen solubility parameters (the X axis) obtained through experiments and Hansen solubility parameters (the Y axis) derived from the machine learning model ML in terms of H-bond force. As shown in FIG. 13A, FIG. 13B and FIG. 13C, the machine learning model ML may derive the Hansen solubility parameters (the Y axis) that are similar to the Hansen solubility parameters (the X axis) obtained through experiments.

FIG. 14 is a block diagram illustrating the computing system 140 according to an example embodiment. In some embodiments, the method of estimating solubility, which has been described above with reference to the accompanying drawings, may be performed by the computing system 140 of FIG. 14.

The computing system 140 may include a stationary computing system, such as a desktop computer, a workstation, or a server, or a portable computing system such as a laptop computer. Referring to FIG. 14, the computing system 140 may include at least one processor 141, an input/output (I/O) interface 142, a network interface 143, a memory subsystem 144, a storage 145, and a bus 146. The at least one processor 141, the I/O interface 142, the network interface 143, the memory subsystem 144, and the storage 145 may communicate with one another through the bus 146.

The at least one processor 141 may be referred to as a processing unit and may execute a program like a CPU, a GPU, an NPU, or a DSP. For example, the at least one processor 141 may access the memory subsystem 144 through the bus 146 and execute instructions stored in the memory subsystem 144. In some embodiments, the computing system 140 may further include an accelerator as a dedicated hardware device designed to perform a certain function at a high speed. In some embodiments, the machine learning model ML in FIG. 1 may be implemented by an NPU included in the at least one processor 141.

The I/O interface 142 may include an input device, such as a keyboard or a pointing device, and/or an output device such as a display device or a printer, or may provide access to an input device and/or an output device. A user may trigger execution of a program 145_1 and/or loading of data 145_2 through the I/O interface 142, input the input data in FIG. 1, or check the obtained solubility parameters and the calculated solubility.

The network interface 143 may provide access to a network outside the computing system 140. For example, the network may include a plurality of computing systems and communication links. The communication links may include wired links, optical links, wireless links, or other types of links.

The memory subsystem 144 may store the program 145_1 for the method of estimating solubility, which has been described above with reference to the accompanying drawings, or at least part of the program 145_1. The at least one processor 141 may perform at least part of the method of estimating solubility by executing a program (or instructions) stored in the memory subsystem 144. The memory subsystem 144 may include ROM, RAM, or the like.

The storage 145 as a non-transitory storage medium may not lose data stored therein even when power supplied to the computing system 140 is interrupted. For example, the storage 145 may include a non-volatile memory device or a storage medium such as a magnetic tape, an optical disk, or a magnetic disk. The storage 145 may be detachable from the computing system 140. As shown in FIG. 14, the storage 145 may store the program 145_1 and the data 145_2. At least part of the program 145_1 may be loaded to the memory subsystem 144 before being executed by the at least one processor 141. In some embodiments, the storage 145 may store a file written in a programming language. The program 145_1, which is generated from the file by a compiler or the like, or at least part of the program 145_1 may be loaded to the memory subsystem 144. The data 145_2 may include data, e.g., the input data and/or the descriptor(s) generated in FIG. 1, needed to perform the method of estimating solubility, which has been described above with reference to the accompanying drawings. The data 145_2 may also include data, e.g., the solubility parameter and the estimated solubility in FIG. 1, generated by performing the method of estimating solubility, which has been described above with reference to the accompanying drawings. The data 145_2 may include at least one of the training data, the sample descriptors, the sample solubility parameters, and the database generated in FIG. 10.

While the inventive concept(s) of the present disclosure have been particularly shown and described with reference to embodiments thereof, it will be understood that various changes in form and details may be made therein without departing from the spirit and scope of the following claims.

What is claimed is:

1. A method of estimating solubility, the method comprising:
obtaining input data representing a chemical structure of a target material;
generating at least one descriptor based on the input data;
obtaining at least one solubility parameter by providing the at least one descriptor to a machine learning model trained based on chemical structures and sample solubility parameters of sample materials; and
calculating the solubility based on the at least one solubility parameter,
wherein the at least one descriptor includes at least one of a zero-dimensional descriptor, a one-dimensional descriptor, a two-dimensional descriptor, or a three-dimensional descriptor, each representing the chemical structure of the target material.

2. The method of claim 1, wherein the zero-dimensional descriptor includes at least one of an atom count, a bond count, atomic charges, atom-centered fragment charges, a total positive charge, a total negative charge, a number of atomic positive charges, a number of atomic negative charges, electronegativity, and ionization potential.

3. The method of claim 1, wherein the one-dimensional descriptor includes at least one of a fragment count, a hydrogen bond acceptor, a hydrogen bond donor, atom-centered fragment charges, and a number of disconnected fragments.

4. The method of claim 1, wherein the two-dimensional descriptor includes at least one of graph invariants, a number of fragment positive charges, a number of fragment negative charges, topological charge indices corresponding to charge transfers between pairs of atoms, and connectivity indices.

5. The method of claim 1, wherein the three-dimensional descriptor includes at least one of a size, a surface, and a volume of the target material.

6. The method of claim 1, wherein the target material includes an ionic compound, and
the at least one descriptor includes a descriptor including information about a charge of the ionic compound.

7. The method of claim 1, wherein the input data corresponds to a string including a series of characters defining the chemical structure of the target material, and
the at least one descriptor is constituted of at least one number.

8. The method of claim 1, wherein the at least one solubility parameter includes a dispersion force parameter, a polar force parameter, and a hydrogen bond force parameter as Hansen solubility parameters.

9. The method of claim 1, wherein the calculating of the solubility includes calculating solubility of a solute in a solvent based on at least one first solubility parameter corresponding to the solute and at least one second solubility parameter corresponding to the solvent.

10. The method of claim 9, wherein, when the solute is a composite of at least two materials, the obtaining of the at least one solubility parameter includes
calculating the at least one first solubility parameter based on a weighted sum of at least two solubility parameters respectively corresponding to the at least two materials,
wherein a weight of the weighted sum corresponds to a proportion of a mass or a volume of each of the at least two materials in the composite.

11. The method of claim 9, wherein, when the solvent is a mixture of at least two solvents, the obtaining of the at least one solubility parameter includes calculating the at least one second solubility parameter based on a weighted sum of at least two solubility parameters respectively corresponding to the at least two solvents,
wherein a weight of the weighted sum corresponds to a proportion of a mass or a volume of each of the at least two solvents in the mixture.

12. The method of claim 1, further comprising generating the trained machine learning model,
wherein the generating of the trained machine learning model includes:
obtaining training data with respect to an attribute of a sample material;
generating a plurality of sample descriptors based on the training data;
extracting at least one sample solubility parameter of the sample material from the training data; and
training the machine learning model based on the plurality of sample descriptors and the at least one sample solubility parameter.

13. The method of claim 12, wherein the training of the machine learning model includes:
identifying importance levels of the plurality of sample descriptors based on the trained machine learning model; and
setting a descriptor feature group based on the importance levels of the plurality of sample descriptors, and
the at least one descriptor is included in the descriptor feature group.

14. The method of claim 12, wherein the training of the machine learning model is based on regression learning using at least one of a random forest and a Gaussian process.

15. A system comprising:
at least one processor; and
a non-transitory storage medium storing instructions allowing the at least one processor to perform operations for solubility estimation when the instructions are executed by the at least one processor,
wherein the operations include:
an operation of obtaining input data representing a chemical structure of a target material;
an operation of generating at least one descriptor based on the input data;
an operation of obtaining at least one solubility parameter by providing the at least one descriptor to a machine learning model trained based on chemical structures and sample solubility parameters of sample materials; and
an operation of calculating the solubility based on the at least one solubility parameter,
wherein the at least one descriptor includes at least one of a zero-dimensional descriptor, a one-dimensional descriptor, a two-dimensional descriptor, or a three-dimensional descriptor, each representing the chemical structure of the target material.

16. The system of claim 15, wherein the target material includes an ionic compound, and
the at least one descriptor includes a descriptor including information about a charge of the ionic compound.

17. The system of claim 15, wherein the input data corresponds to a string including a series of characters, and
the at least one descriptor is constituted of at least one number.

18. A method of estimating solubility, the method comprising
generating a machine learning model trained to derive at least one solubility parameter from at least one descriptor defining a chemical structure of a material,
wherein the generating of the trained machine learning model includes:
obtaining training data with respect to an attribute of a sample material;
generating a plurality of sample descriptors based on the training data;
extracting at least one sample solubility parameter of the sample material from the training data; and
training the machine learning model based on the plurality of sample descriptors and the at least one sample solubility parameter,
wherein the plurality of sample descriptors include at least one of a zero-dimensional descriptor, a one-dimensional descriptor, a two-dimensional descriptor, or a three-dimensional descriptor, each representing a chemical structure of the sample material.

19. The method of claim 18, wherein the sample material includes an ionic compound, and
the plurality of sample descriptors include a descriptor including information about a charge of the ionic compound.

20. The method of claim 18, wherein the training of the machine learning model is based on regression learning using at least one of a random forest and a Gaussian process.
